(19) United States
(12) Patent Application Publication
(10) Pub. No.: US 2004/0106197 A1
(43) Pub. Date: Jun. 3, 2004

(54) Central nerve system precursor cells inducing synaptogenic neurons in spinal cord

(76) Inventors: Hideyuki Okano, Suita-shi (JP); Yuhto Ogawa, Kawasa-shi (JP)

Correspondence Address: FOLEY AND LARDNER, SUITE 500, 3000 K STREET NW, WASHINGTON, DC, 20007, US

(21) Appl. No.: 10/472,531

(22) PCT Filed: Nov. 2, 2001
(86) PCT No.: PCT/JP01/09620

(30) Foreign Application Priority Data
Mar. 28, 2001 (JP) 2001-93881

(51) Int. Cl. C12N 5/08

(52) U.S. Cl. 435/368

(57) ABSTRACT

The present invention provides central nervous system neural progenitor cells which can induce neurons with synapse forming ability, oligodendrocytes, astrocytes and the like when transplanted into an injured or disabled spinal cord, a method for preparing said central nervous system neural progenitor cells, a method for screening promoters or inhibitors of synapse formation using said central nervous system neural progenitor cells, a therapeutic drug to improve neural injuries or neural functions using said central nervous system neural progenitor cells, and the like. The central nervous system neural progenitor cells comprising neural stem cells derived from the spinal cord and cultured in the presence of cytokine, is transplanted into the injury site at a certain period after the spinal injury. The transplantation can induce neurons with synapse forming ability, oligodendrocytes, and astrocytes in the injury site, resulting in forming synapses between induced neurons and host neurons, and thus the injured spinal cord function is improved.

### TECHNICAL FIELD

The present invention relates to central nervous system neural progenitor cells (CNS-NPCs) which can induce neurons or the like with synapse forming ability in a spinal cord, a method for preparing said central nervous system neural progenitor cells, and a method for screening promoters or inhibitors of synapse formation using said central nervous system neural progenitor cells and the like.

### BACKGROUND ART

A spinal cord injury is a disease wherein spinal code tissues are injured by trauma and the spinal function is damaged. As therapies for this disease, high dose steroid therapies in order to minimize chemical secondary injury occurring immediately after the trauma, rehabilitation therapies in order to maximize the remaining functions, and surgical therapies such as a muscle transfer have been performed to date. It has been reported that, among steroid drugs, high dose of methylprednisolone is effective to improve neurologic symptons associated with the spinal cord injury (J.Spinal Disord. 5(1), 125-131,1992). However, beside high dose of steroid drug causes strong systemic side effect and is difficult to control, it also has a problem of decreasing the protective function in the spinal cord injury involving infectious diseases. Further, even the effectiveness of high dose steroid therapy is now under discussion. As to therapies for the spinal cord injury, therapies to minimize tissue damages at the acute period and therapies to maximize the remaining functions are currently performed. A therapy has not yet been established, however, to regenerate injured spinal cords in the adult spinal cord where regeneration of lost neurons by damage or re-elongation of ruptured axons are unobserved.

Other reported therapies for the spinal cord injury include a method for transplanting a therapeutically effective amount of neural astrocytes pretreated in vitro with inflammation-associated cytokines, into an injury site in the central nervous system (CNS) (published Japanese translation of a PCT application No. 2000-503983), a method for promoting the regeneration of neural axons in a mammal CNS by administering mononuclear phagocytes (such as monocytes and macrophages) of the same species to the injured or diseased site, or to the central nervous system (CNS) in its vicinity (J. Mol. Med. 77, 713-717, 1999; J. Neurosci. 19(5), 1708-16, 1999; Neurosurgery 44(5), 1041-1045, 1999; Trends. Neurosci. 22(7), 295-299, 1999)(published Japanese translation of PCT application No. 11-513370), and so on. Although the definite mechanism is unknown, it has also been reported that vaccination with spinal cord homogenates or administration of T cells specific to myelin basic protein, which is a marrow sheath protein, promoted the recovery of exercise endurance after the spinal cord injury (Neuron 24, 639-647, 1999; Lancet 354, 286-287, 2000).

On the other hand, as to transplant experiments on the injured spinal cord using cultured cells, it has been reported that improvement in the function was found, when central nervous system neural progenitor cells (CNS-NPCs) differentiated from mouse ES cells were transplanted into a spinal cord injury model rat (Nat. Med. 5, 1410-1412, 1999). However, this method has an ethical problem in the point that it uses ES cells, and the induction by differentiation from ES cells into CNS-NPCs has not yet been fully established, wherein the generation of teratomas at the transplant site is considered. In addition, the experiment wherein the regeneration of the spinal cord is aimed and the fetal spinal cord is transplanted, has been performed by using spinal cord injury model rats and cats, and improvement in the function of injured spinal cord by transplantation has been reported (J. Neurosci. 18, 763-778, 1998; Brain Pathol. 5, 451-457, 1995 and others), however, such treatment has not yet been established for clinical application. One underlying reason is the difficulty to secure donor fetal spinal cords, as spinal cords from a number of fetuses are required for one transplantation.

Central nervous system injuries including the spinal cord injury are diseases which are very difficult to treat, without effective therapies by now as mentioned above, and the development of new therapies is strongly expected. Further, with the recent increase of auto accidents and aging population, the number of patients who suffer from the spinal cord injury tends to increase, which is becoming a big social issue. An object of the present invention is to provide central nervous system neural progenitor cells which can induce neurons with synapse forming ability, oligodendrocytes, astrocytes and the like by transplanting said central nervous system neural progenitor cells into an injured or disabled spinal cord, a method for preparing said central nervous system neural progenitor cells, a method for screening promoters or inhibitors of synapse formation using said central nervous system neural progenitor cells or the like, a therapeutic drug to improve a neural damage or a neural function using said central nervous system neural progenitor cells, and the like.

To attain the above-mentioned object, the inventors of the present invention cultured fetal rat spinal cord tissues at embryonic day 14.5 and obtained central nervous system neural progenitor cells (CNS-NPCs) according to the method as described previously (Science 255, 1707-1710, 1992). It has been discovered that the direct injection of the above-mentioned central nervous system neural progenitor cells into a spinal cord injury model rat at the injury site 9 days after the injury was able to induce neurons, oligodendrocytes, astrocytes and the like derived from transplanted cells at the injury site, further form myelins in the neuronal axons derived from said transplanted cells, and improve the function of the spinal cord by forming synapses. The present invention has thus been completed.

### DISCLOSURE OF THE INVENTION

The present invention relates to central nervous system neural progenitor cells which can induce neurons with synapse forming ability, in a spinal cord (claim 1), central nervous system neural progenitor cells which can induce oligodendrocytes and/or astrocytes in addition to neurons with synapse forming ability, in a spinal cord (claim 2), the central nervous system neural progenitor cells according to claim 1 or 2, wherein the spinal cord is an injured spinal cord (claim 3), and the central nervous system neural progenitor cells according to any of claims 1 to 3, wherein the spinal cord is a human spinal cord (claim 4).

The present invention also relates to a method for preparing central nervous system neural progenitor cells which can induce neurons with synapse forming ability, in a spinal cord, wherein neural stem cells derived from a spinal cord is cultured in the presence of cytokine (claim 5), a method for preparing central nervous system neural progenitor cells which can induce oligodendrocytes and/or astrocytes in addition to neurons with synapse forming ability, in a spinal cord, wherein neural stem cells derived from a spinal cord is cultured in the presence of cytokine (claim 6), the method for preparing the central nervous system neural progenitor cells according to claim 5 or 6, which can be induced in an injured spinal cord (claim 7), the method for preparing the central nervous system neural progenitor cells according to any of claims 5 to 7, which can be induced in a human spinal cord (claim 8), the method for preparing the central nervous system neural progenitor cells according to any of claims 5 to 8, wherein the cytokine is a basic fibroblast growth factor (claim 9), and the method for preparing the central nervous system neural progenitor cells according to any of claims 5 to 9, wherein neural stem cells derived from a human spinal cord is used (claim 10).

The present invention further relates to a method for screening promoters or inhibitors of synapse formation, wherein the central nervous system neural progenitor cells according to any of claims 1 to 4 or neurons induced from said cells are made to contact with a subject material, at least in a spinal cord, and the level of synapse formation in neurons induced from said central nervous system neural progenitor cells are evaluated (claim 11), promoters of synapse formation obtained by the method for screening the promoters or the inhibitors of synapse formation according to claim 11 (claim 12), inhibitors of synapse formation obtained by the method for screening the promoters or the inhibitors of synapse formation according to claim 11 (claim 13), a therapeutic drug to improve a neural injury or a neural function, wherein the central nervous system neural progenitor cells according to any of claims 1 to 4 are used as an active component (claim 14), a therapeutic drug to improve a neural injury or a neural function, wherein the central nervous system neural progenitor cells according to any of claims 1 to 4 and the promoters of synapse formation according to claim 12 are used as active components (claim 15), a therapeutic method to improve a neural injury or a neural functional disease, wherein the therapeutic drug to improve the neural injury or the neural function according to claim 14 or 15 is introduced into a spinal cord (claim 16), a therapeutic method to improve a neural injury or a neural functional disease, wherein the central nervous system neural progenitor cells according to any of claims 1 to 4 is introduced into a spinal cord by transplantation (claim 17), a method for inducing any of neurons, oligodendrocytes, or astrocytes in a spinal cord, wherein the central nervous system neural progenitor cells according to any of claims 1 to 4 are transplanted into a spinal cord (claim 18), and a synapse formed in a neuron, which is induced by transplanting the central nervous system neural progenitor cells according to any of claims 1 to 4 into a spinal cord (claim 19).

### BRIEF DESCRIPTION OF DRAWINGS

FIG. 1 explains a method for constructing a spinal cord injury model rat at the cervical vertebra level by weight compression method.

FIG. 2 is a picture showing the differentiation of transplanted neural stem cells in the host spinal cord.

FIG. 3 shows (a) a method for testing the forelimb skilled behavior after the transplant of neural stem cells and (b) the recovery result thereof.

FIG. 4 is a picture showing the differentiation into neurons and synapse formation of donor cells in the host spinal cord.

FIG. 5 is a picture showing the survival of the transplanted human neural stem cells in the host spinal cord injury rat.

### BEST MODE OF CARRYING OUT THE INVENTION

As to the central nervous system neural progenitor cells of the present invention, there is no particular limitation, as far as they are central nervous system neural progenitor cells derived from vertebrates, which can induce neurons with synapse forming ability, preferably oligodendrocytes, astrocytes and the like in addition to said neurons with synapse forming ability, in the spinal cord, especially in the spinal cord of vertebrates such as human, wherein the spinal cord is injured. As the above-mentioned vertebrates, specific examples include vertebrates such as human, rat, mouse, cat, monkey, goat, rabbit, dog, cattle, sheep, zebrafish, cyprinodont, shark, frog and the like, but are not limited to these examples. Further, when the central nervous system neural progenitor cells are human central nervous system neural progenitor cells, it is more preferable to prepare the cells from spinal cords derived from aborted fetuses, in respect that cells for transplantation can be obtained without limitation and the problem of the shortage of donor can be resolved.

In the present invention, as to a method for preparing the central nervous system neural progenitor cells which can induce neurons with synapse forming ability, and the central nervous system neural progenitor cells which can induce oligodendrocytes and/or astrocytes in addition to neurons with synapse forming ability, in the spinal cord, preferably in the injured spinal cord, it is not limited in particular as far as it is a method for culturing neural stem cells derived from a spinal cord in the presence of cytokine. The neural stem cell derived from a spinal cord, which was cultured in the presence of cytokine, can be induced to neurons, oligodendrocytes and astrocytes in the injured spinal cord, by introduction and transplantation into the injured spinal cord. Further, origins of the injured spinal cord and the neural stem cell may be same or different, however, it is preferable to use the injured spinal cord derived from human and the neural stem cells derived from a human spinal cord. For example, neural stem cells derived from the human spinal cord can be introduced/transplanted into the injured spinal cord of a rat. When using neural stem cells derived from the human spinal cord, it is preferable to use neural stem cells derived from the spinal cord of aborted human fetuses.

As to the method for culturing the neural stem cells derived from the spinal cord in the presence of cytokine, it is not limited in particular, and a suitable example is a method of floating culture, wherein a collected spinal cord is treated with trypsin by ordinary methods followed by the dispersion of cells by pipetting and the like, which are then floating cultured for 7 to 10 days at 37° C. by Neurosphere method, a selective culturing method for neural stem cells (Science 255, 1707-1710, 1992). Cell aggregates called Neurosphere, a cell population richly containing neural stem cells, can be obtained by this floating culture method. Sufficient amount of neural stem cells for transplantation can be secured, by dissociating the Cladophora sauteri like Neurosphere into single cells one by one by pipetting and the like, and repeating subculture two to four times to obtain Neurospheres by floating culture again under the same condition. As a culture solution for floating culture, a serum-free DMEM/F12 medium is preferable, and as a cytokine used for the above-mentioned culture solution, IL-12, IL-1α, IL-1β, IFN-γ, TNF-α, FGF-2, GM-CSF, IL-4 and the like can be specifically exemplified, or the combination of one or more species selected from the above-mentioned cytokines may be used, but among them, FGF-2 (basic fibroblast growth factor) is preferable. Further, in conjunction with cytokine, EGF (epidermal growth factor), NGF (nerve growth factor), PDGF (platelet derived growth factor), neuropeptide, leukemia inhibitory factor and the like may also be used.

Promoters or inhibitors of synapse formation can be screened with the use of the central nervous system neural progenitor cells of the present invention. As said method for screening promoters or inhibitors of synapse formation, for example, a method can be given, wherein the central nervous system neural progenitor cells of the present invention or neurons induced from said cells are made to contact with a subject material, at least in the spinal cord, and the level of synapse formation in the neurons induced from the above-mentioned central nervous system neural progenitor cells is evaluated. As to the method for contacting the above-mentioned central nervous system neural progenitor cells or the neurons induced from said cells with a subject material, specific examples include a method for transplanting a mixture of the central nervous system neural progenitor cells and a subject material into an injured spinal cord, a method for transplanting the central nervous system neural progenitor cells into an injured spinal cord after oral administration of a subject material, a method wherein the central nervous system neural progenitor cells are transplanted into an injured spinal cord, and a subject material is injected into an induced neuron, and so on. Further, as to the method for evaluating the level of synapse formation, specific examples include methods such as electronmicroscopy, immunohistological analysis for synaptophysin or the like. As the promoters of synapse formation obtained by said screening method, for example, BDNF, NT-3, NGF and the like can be specifically exemplified, and as the inhibitors of synapse formation, semaphorin, Nogo, MAG and the like can be exemplified. The promoters or inhibitors of synapse formation of the present invention, however, means a material whose action to promote or inhibit synapse formation has not been known to date.

As a therapeutic drug to improve neural injuries or neural functions of the present invention, it can be any therapeutic drug as far as it contains the above-mentioned central nervous system neural progenitor cells as an active component, or the above-mentioned central nervous system neural progenitor cells and the promoters of synapse formation as described above as active components. When said central nervous system neural progenitor cells or promoters of synapse formation is used as a therapeutic drug for neural injuries or neurologic dysfunctional diseases, various prescriptional ingredients which are pharmaceutically permitted, such as a regular carrier, immunosuppressive drug, binding agent, stabilizing agent, excipient, diluent, pH buffer agent, disintegrant, solubilizer, solubilizing adjuvant, isotonic agent and the like can be added. Further, said therapeutic drug, for example, can be administered parenterally to a spot such as the injured site of the spinal cord by injecting a dosage form such as a solution, emulsion, suspension or the like.

As a therapeutic method to improve neural injuries or neural functional diseases of the present invention, a method for introducing the therapeutic drug to improve neural injuries or neural functions as described above into the spinal cord, or a method for injecting/transplanting the above-mentioned central nervous system neural progenitor cells into the spinal cord can be exemplified. With the use of said therapeutic method, synapse formation in the neurons induced from the central nervous system neural progenitor cells emerges, and can improve neural injuries or neural functional diseases. Further, a method for inducing any of neurons, oligodendrocytes or astrocytes into the spinal cord of the present invention means a method for transplanting the central nervous system neural progenitor cells of the present invention by direct injection into the spinal cord, which can induce neurons, oligodendrocytes and astrocytes, which are main cells constituting the central nervous system, into the spinal cord tissues of the injury site. The present invention also relates to synapses formed in neurons induced by transplanting the central nervous system neural progenitor cells of the present invention into the spinal cord. Said synapse formation can improve spinal cord functions injured by damage.

The present invention will now be explained in more detail with examples, but the technical scope of the present invention is not limited to these exemplifications.

### REFERENCE EXAMPLE 1

### Preparation of Cells for Transplantation Derived from Fetal Rat Spinal Cords

Spinal cords were collected from Spraque-Dawley rat embryos at embryonic day 14.5 and were tripsinized according to usual methods. Cells were dispersed by pipetting, which were then cultured by Neurosphere method, a selective culturing method for neural stem cells. The culture as described above was performed using a non-serum DHEM/F12 medium containing a basic fibroblast growth factor (FGF-2) as a growth factor, with said cells floating cultured for a week at 37° C., and cell aggregates called Neurosphere, a cell population richly containing neural stem cells, were obtained. This Neurosphere was dispersed into individual cells one by one by pipetting, which was floating cultured again under the same condition to obtain Neurospheres. Said subculture was repeated for 2 to 4 times to obtain sufficient amount of neural stem cells for transplantation. The obtained cells were labeled with Bromodeoxyuridine (BrdU), a fluorescent substance that generates red fluorescent.

### REFERENCE EXAMPLE 2

### Transplantation of Neural Stem Cells into Spinal Cord Injury Model Rats

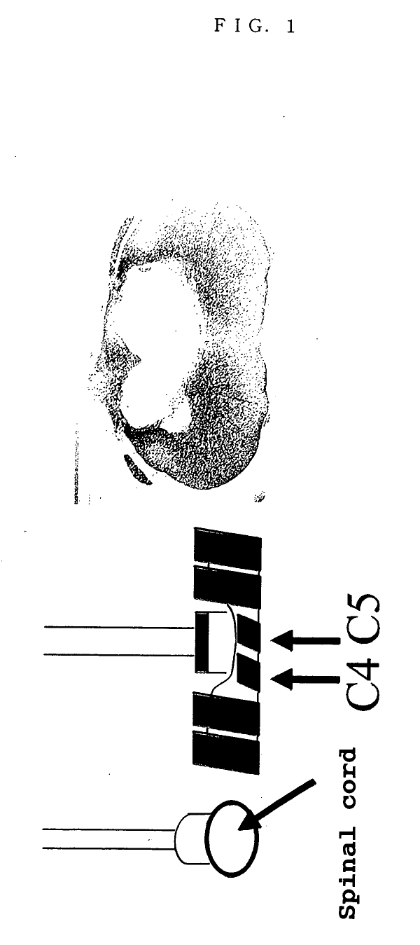

Adult spinal cord injury model rats (female SD rats, body weight 200 to 230 g) were constructed using weight compression method according to Holtz et al. (Surg. Neurol. 31, 350-360, 1989). To be specific, adult spinal cord injury model rats were constructed by performing a laminectomy of the forth and fifth cervical vertebrae (C4, C5) and by compressing the spinal cord with a 35 g weight stationed on the high site of the forth and fifth cervical vertebrae from the dorsal spinal cord for 15 minutes (see FIG. 1: reference picture 1). Nine days after the injury, the neural stem cells were transplanted by injection of 30 μl of culture containing 5-10×10⁶/ml of neural stem cells obtained in the Reference Example 1, into a cavity occurred at the spinal cord injury site with a microsyringe under an operating microscope.

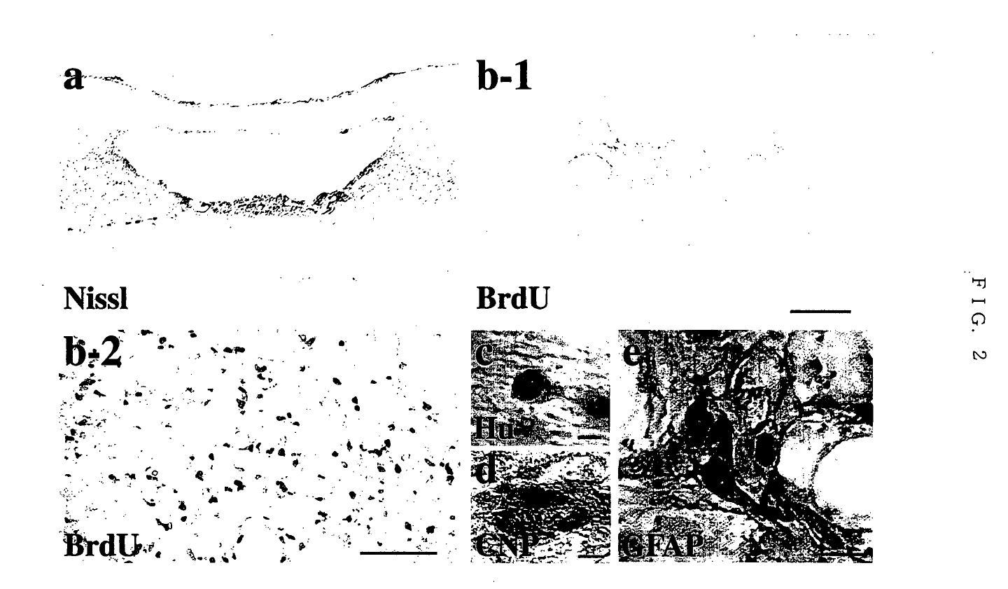

Five weeks after the transplantation, the transplanted rats were perfusion fixed with paraformaldehyde, and the transplanted spinal cord was then removed to be analyzed histologically. The results are shown in FIG. 2 (see reference picture 2). FIG. 2a shows the injury site of the spinal cord injury animal transplanted only with culture medium, indicating a cavity formed by the injury. FIG. 2b-1 shows the injury site of the spinal cord injury animal transplanted with neural stem cells, which had been pre-labeled with BrdU (scale bar 250 μm), FIG. 2b-2 is a enlarged picture of b-1 (scale bar 100 μm). FIG. 2c shows donor cells differentiated into neurons (brown: neuron marker Hu, gray: BrdU), FIG. 2d shows donor cells differentiated into oligodendrocytes (brown: oligodendrocyte marker CNP, gray: BrdU), and FIG. 2e shows donor cells differentiated into astrocytes (brown: astrocyte marker GFAP, gray: BrdU). These results confirmed the existence of neurons, oligodendrocytes and astrocytes derived from transplanted cells at the transplant site. To confirm neurons, oligodendrocytes and astrocytes, an anti-Hu antibody, anti-2′3′-cyclic nucleotide 3′-phosphohydrolase antibody and anti-Glial fibrillary acidic protein antibody were used, respectively. Further, differentiated cells into neurons, oligodendrocytes or astrocytes were confirmed to be derived from transplanted neural stem cells by Bromodeoxyuridine label.

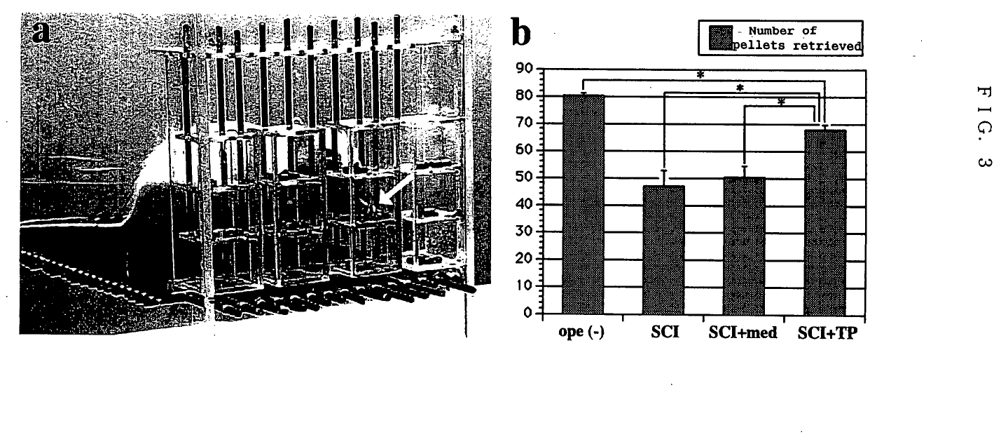

On the other hand, functional evaluation of rats regarding the behavior of grasping a small pellet and carrying it to their mouths with forelimbs was conducted 5 weeks after the transplantation (see FIG. 3: reference picture 3). FIG. 3a shows the design of pellet retrieval test, consisting of 2.5 cubic cm boxes arranged in 4 rows and 3 columns, comparted with iron bars which force rats to retrieve small food pellets in the boxes only with their forelimbs. Five pieces of pellets were placed in each box, and the number of pellets retrieved in 15 minutes was counted. The protocol of said test is as follows: rats were pre-trained with limited access to food for a week; then operated with the above-mentioned weight compression method; pre-trained in the same manner for 4 weeks after the transplantation; and tested for 2 consecutive days at 5 weeks after the transplantation. The results are shown in FIG. 3b. As shown in FIG. 3b, the result of control animal group without spinal cord injury (ope(−); n=10) was 80.30±0.84, that of the group with spinal cord injury but without transplantation (SCI; n=8) was 47.12±5.76, that of the group with spinal cord injury and transplanted only with culture medium (SCI+med; n=9) was 50.11±4.19, and that of the group with spinal cord injury and transplanted with neural stem cells (SCI+TP; n=13) was 67.85±2.02. A statistically significant functional improvement was observed in the transplanted group compared to the control groups without transplantation (Mann-Whitney U-test). These results of skilled behavior test of forelimbs confirmed that the functional improvement was observed by the transplantation.

### Example 1

### Confirmation of the Induction of Neurons Derived from Transplanted Cells into Host Neural Network, in Transplant Experiments of CNS-NPCs Derived from Fetal Rat Spinal Cords into Spinal Cord Injury Model

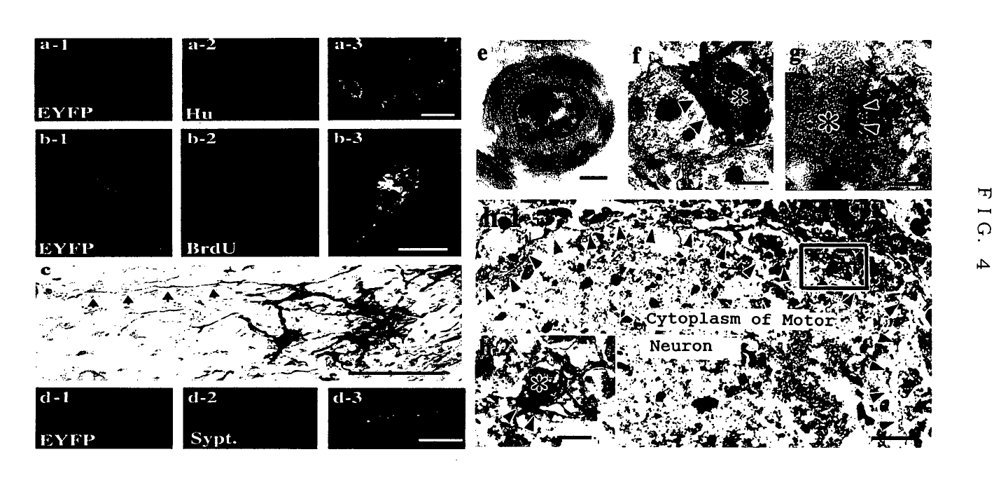

Cells for transplantation derived from transgenic rats which express EYFP (enhanced yellow fluorescent protein) specifically in neurons, were prepared and transplanted in the same manner as in Reference Example 2. Five weeks after the transplantation, the spinal cord at the transplant site was collected. The above-mentioned transgenic rats expressing EYFP were constructed according to the method as described previously (Sawamoto et al. J. Neurosci. in press). Briefly, EYFPcDNA under the control of a 1.1-kb promoter factor of the Tα-1 tubulin gene expressed in the nervous system was purified by the method as described previously (J. Neurosci. 14, 7319-7330, 1994); this cDNA was microinjected into a pronucleus of a rat fertilized egg, which was then returned to a SD rat, a tentative parent and thus a transgenic rat was constructed. Said transgenic rat was extracted a genome DNA from its tail and identified by PCR using a primer specific to the introduced EYFPcDNA. Differentiation/existence of neurons derived from transplanted cells in the host spinal cord, was confirmed by staining the spinal cord at the transplant site with an anti-EYFP antibody. The results are shown in FIGS. 4a-d (see reference picture 4). FIG. 4a shows that all cells expressing EYFP are Hu-positive neurons (scale bar 5 μm). FIG. 4b shows the state in which donor cells were divided and differentiated into neurons after the transplantation in the host spinal cord (scale bar 5 μm). In FIG. 4c, neurons derived from EYFP-positive donors were observed to extend their axons longitudinally in the host spinal cord (scale bar 50 μm). FIG. 4d shows an accumulation of synaptophysin-positive synapse vesicles in the periphery of neurons derived from EYFP-positive donors (scale bar 5 μm).

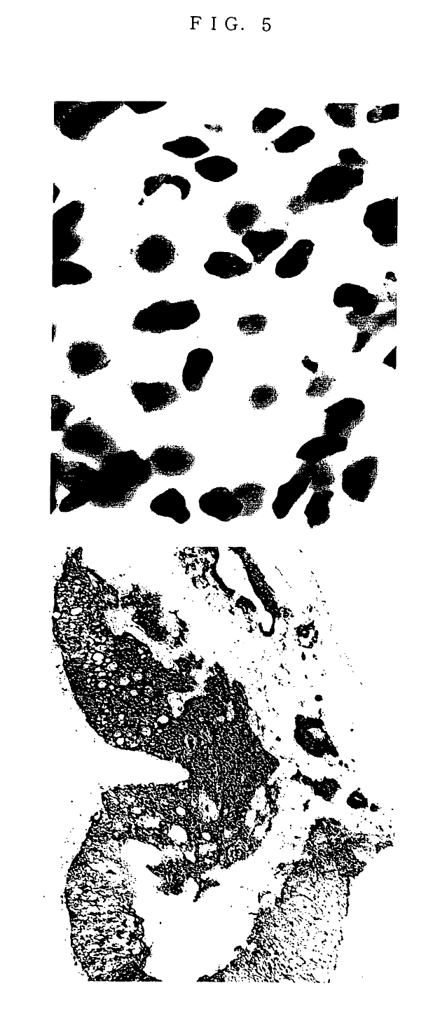

Further, the result examined with an electron microscope after staining this tissue with anti-EYFP antibody is shown in FIGS. 4e-h (see reference picture 4). FIG. 4e shows an immuno electronmicroscopic analysis, indicating that some axons of neurons derived from EYFP-positive donors were myelinated in the host spinal cord partially (scale bar 0.1 μm). FIG. 4f shows that neurons derived from EYFP-positive donors, acting as presynaptic cells, form synapses with host neurons (*) (scale bar 0.5 μm). FIG. 5g shows that neurons derived from EYFP-positive donors, acting as postsynaptic cells, form synapses with host neurons (*) (scale bar 0.2 μm). FIG. 5h shows that host motor neurons at the injury level and neurons derived from EYFP-positive donors form synapses (scale bar h-1: 2 μm, h-2: 0.5 μm). FIGS. 4e-h show myelin formations on axons of neurons derived from EYFP-positive cells, in other words, transplanted cells, and confirmed synapse formations between neurons derived from transplanted cells and EYFP-negative neurons, in other words, host neurons. These results confirmed that neural stem cells used for transplantation were central nervous system neural progenitor cells, which can induce oligodendrocytes and/or astrocytes in addition to neurons with synapse forming ability, in the spinal cord.

### Example 2

### Transplant Experiments of CNS-NPCs Derived from Human Aborted Fetuses into Spinal Cord Injury Model Rats

Spinal cords were collected from human aborted fetuses at embryonic week 9, and cultured to obtain sufficient amount of cells for transplantation in the same manner as in Reference Example 1, except using FGF-2 and epidermal growth factor (EGF) as growth factors, and a medium added with leukemia inhibitory factor. As to spinal cord injury model rats, they were constructed by the method of Tator with a 35 g of compression (J. Neuropathol. Exp. Neurol. 58, 489-498, 1999), and 9 days after the injury, injected with the cell for transplantation obtained by the method described above into a cavity occurring at the spinal cord injury site using a microsyringe under an operating microscope. In addition, said spinal cord injury model rats had been administered intraperitoneally with 10 μg/g of body weight of Cyclosporine A as an immunosuppressive drug, every day from the previous day of the transplantation. Five weeks after the transplantation, the transplanted cells were confirmed to survive at the transplant site, by staining the spinal cord transplant site with an antibody against anti-human nuclear specific antigen (see FIG. 5: reference picture 5).

### Industrial Applicability

The present invention makes it possible to induce neurons with synapse forming ability, oligodendrocytes, and astrocytes by transplanting central nervous system neural progenitor cells derived from a spinal cord into an injured spinal cord, which results in improving the injured spinal cord function with an experiment using a spinal cord injury model rat. The present invention also makes it possible to transplant cultured central nervous system neural progenitor cells derived from a human fetal spinal cord into a spinal cord injury model rat and integrate said cells there. It is expected that expansion of these technologies can lead to development of new therapies in attempts to regenerate the spinal cord for the injured spinal cord.

1. Central nervous system neural progenitor cells which can induce neurons with synapse forming ability, in a spinal cord.

2. Central nervous system neural progenitor cells which can induce oligodendrocytes and/or astrocytes in addition to neurons with synapse forming ability, in a spinal cord.

3. The central nervous system neural progenitor cells according to claim 1 or 2, wherein the spinal cord is an injured spinal cord.

4. The central nervous system neural progenitor cells according to any of claims 1 to 3, wherein the spinal cord is a human spinal cord.

5. A method for preparing central nervous system neural progenitor cells which can induce neurons with synapse forming ability, in a spinal cord, wherein neural stem cells derived from a spinal cord is cultured in the presence of cytokine.

6. A method for preparing central nervous system neural progenitor cells which can induce oligodendrocytes and/or astrocytes in addition to neurons with synapse forming ability, in a spinal cord, wherein neural stem cells derived from a spinal cord is cultured in the presence of cytokine.

7. The method for preparing the central nervous system neural progenitor cells according to claim 5 or 6, which can be induced in an injured spinal cord.

8. The method for preparing the central nervous system neural progenitor cells according to any of claims 5 to 7, which can be induced in a human spinal cord.

9. The method for preparing the central nervous system neural progenitor cells according to any of claims 5 to 8, wherein the cytokine is a basic fibroblast growth factor.

10. The method for preparing the central nervous system neural progenitor cells according to any of claims 5 to 9, wherein neural stem cells derived from a human spinal cord is used.

11. A method for screening promoters or inhibitors of synapse formation, wherein the central nervous system neural progenitor cells according to any of claims 1 to 4 or neurons induced from said cells are made to contact with a subject material, at least in a spinal cord, and the level of synapse formation in neurons induced from said central nervous system neural progenitor cells are evaluated.

12. Promoters of synapse formation obtained by the method for screening the promoters or the inhibitors of synapse formation according to claim 11.

13. Inhibitors of synapse formation obtained by the method for screening the promoters or the inhibitors of synapse formation according to claim 11.

14. A therapeutic drug to improve a neural injury or a neural function, wherein the central nervous system neural progenitor cells according to any of claims 1 to 4 are used as an active component.

15. A therapeutic drug to improve a neural injury or a neural function, wherein the central nervous system neural progenitor cells according to any of claims 1 to 4 and the promoters of synapse formation according to claim 12 are used as active components.

16. A therapeutic method to improve a neural injury or a neural functional disease, wherein the therapeutic drug to improve the neural injury or the neural function according to claim 14 or 15 is introduced into a spinal cord.

17. A therapeutic method to improve a neural injury or a neural functional disease, wherein the central nervous system neural progenitor cells according to any of claims 1 to 4 is introduced into a spinal cord by transplantation.

18. A method for inducing any of neurons, oligodendrocytes, or astrocytes in a spinal cord, wherein the central nervous system neural progenitor cells according to any of claims 1 to 4 are transplanted into a spinal cord.

19. A synapse formed in a neuron, which is induced by transplanting the central nervous system neural progenitor cells according to any of claims 1 to 4 into a spinal cord.
